(19) United States
(12) Patent Application Publication
(10) Pub. No.: US 2020/0352880 A1
(43) Pub. Date: Nov. 12, 2020

(54) USE OF MEMANTINE (MEM) IN PREVENTION AND/OR TREATMENT OF DISEASES CAUSED BY MULTIDRUG-RESISTANT AND NON-RESISTANT BACTERIAL INFECTIONS

(71) Applicant: Sheng-He Huang, Arcadia, CA (US)

(72) Inventor: Sheng-He Huang, Arcadia, CA (US)

(21) Appl. No.: 16/870,016

(22) Filed: May 8, 2020

(30) Foreign Application Priority Data
May 8, 2019 (CN) 201910379111.0

(51) Int. Cl. A61K 31/13; A61K 31/43; A61P 31/04

(52) U.S. Cl. CPC A61K 31/13; A61P 31/04; A61K 31/43

(57) ABSTRACT

A method for applying memantine (MEM) in preparation of one or more drugs for preventing or treating a disease caused by multidrug-resistant or non-resistant bacterial infections is disclosed. MEM performs an antibacterial or bactericidal action through neutrophils and neutrophil extracellular traps (NETs) to prevent and treat diseases caused by multidrug-resistant or non-resistant bacterial infections. A combined use of MEM and an antibiotic in preparation of drugs for prevention and treatment of diseases caused by multidrug-resistant or non-resistant bacterial infections is also disclosed. By applying cell invasion, cell migration, bacteria-infected mice, Next Generation Sequencing, and other techniques, the antibacterial mechanism of MEM is studied using cell models (in vitro), mouse models (in vivo), and NETs, MEM may effectively block bacteremia/sepsis and diseases caused by multidrug-resistant or non-resistant bacterial infections including bacterial meningitis by inhibition of alpha7 nicotinic acetylcholine receptor (α7nAChR).

### CROSS-REFERENCE TO RELATED APPLICATION(S)

The present application claims the benefit of and priority to a Chinese Patent Application Serial No. 201910379111.0, filed on May 8, 2019, (hereinafter referred to as '111.0 application”). The disclosure of the '111.0 application is hereby incorporated fully by reference into the present disclosure for all purposes.

### FIELD

The present disclosure relates to a technical field of pharmaceutical use, in particular to a use of memantine (MEM) in prevention and/or treatment of diseases caused by multidrug-resistant and non-resistant bacterial infections.

### BACKGROUND

The phrase “multidrug-resistant bacteria” does not refer specifically to a particular type of bacteria, but generally refers to the bacteria that are resistant to a variety of antibiotics commonly used today, also known as superbugs. These bacteria have a strong resistance to antibiotics and are able to avoid the risk of being killed. The main superbugs of particular concern are methicillin-resistant Staphylococcus aureus (MRSA), multidrug-resistant Streptococcus pneumoniae (MDRSP), vancomycin-resistant Enterococcus (VRE), multidrug-resistant Mycobacterium tuberculosis (MDR-TB), multidrug-resistant Acinetobacter baumannii (MRAB), newly discovered E. coli and Klebsiella pneumoniae carrying NDM-1 gene, and so on. Since most antibiotics do not work, superbugs have caused great harm to human health. Currently, antibiotic treatments for bacterial infections kill not only pathogenic bacteria but also probiotics in body, often leading to normal flora disturbance/dysbacteriosis, which may cause other diseases and even growth of superbugs. Therefore, development of a new treatment option that eliminates multidrug-resistant bacteria (pathogenic bacteria) without harming probiotics has enormous significance for prevention and control of superbug infections.

Bacterial meningitis has three typical pathogenic hallmark features including nuclear factor-kappa B (NF-κB) activation, bacterial invasion into brain tissue, and leukocyte infiltration. Both bacterial invasion and leukocyte infiltration occur across blood-brain barrier (BBB), which consists primarily of brain microvascular endothelial cells (BMECs). Presently, the main challenges on treatment of bacterial meningitis are how these three pathogenic hallmark features can be effectively intervened during the disease process and whether there is a drug can block these three pathogenic hallmark features concurrently.

Studies have found that alpha7 nicotinic acetylcholine receptor (α7nAChR), an essential regulatory factor for meningitis, is abundantly expressed in hippocampus (the most vulnerable area to meningitis). In α7nAChR knockout (KO) BMECs and mice, E44 invasion and polymorphonuclear neutrophil (PMN) transmigration are significantly decreased, stimulation of nicotine is also blocked, and secretion of proinflammatory cytokines in hippocampus is remarkably reduced. α7nAChR-mediated calcium influx in BMECs caused by bacterial infections is completely obstructed. These findings suggest that α7nAChR may be a promising target for treatment of bacterial meningitis, and all three pathogenic hallmark features of bacterial meningitis may be effectively blocked by inhibiting the function of α7nAChR.

Memantine Hydrochloride, with chemical name 3,5-Dimethyl-1-aminoadamantane hydrochloride, also known as 3,5-Dimethyltricyclo(3.3.1.1(3,7))decan-1-amine hydrochloride, is hereinafter referred to as MEM or memantine. MEM, originally identified as an N-methyl-D-aspartate receptor (NMDAR) antagonist that relieves symptoms in patients with Alzheimer's disease (AD) by blocking the effects of abnormal glutamate activity, is an FDA-approved drug used for treatment of moderate-to-severe AD. The drug is found to have a potent inhibitory effect on α7nAChR (IC50=5.1 μmol/L) that is stronger than the inhibitory effect on NMDAR, explaining MEM is a dual α7nAChR/NMDAR antagonist.

### DETAILED DESCRIPTION

In a first aspect of the present disclosure, a use of memantine (MEM) in preparation of drugs for prevention and/or treatment of diseases caused by bacterial infections is provided, where the chemical structural formula of MEM is shown as (I):

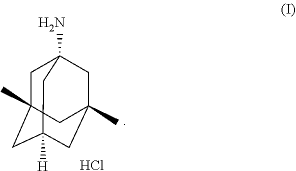

In a second aspect of the present disclosure, a use of MEM in combination with an antibiotic in preparation of drugs for prevention and/or treatment of the diseases caused by bacterial infections is provided, where the chemical structural formula of MEM is shown as the (I) above.

In one implementation of the present disclosure, the diseases caused by bacterial infections are diseases caused by intracellular multidrug-resistant bacterial infections including E. coli E44.

In one implementation of the present disclosure, the disease is bacterial meningitis or neonatal sepsis.

In one implementation of the present disclosure, MEM prevents and/or treats the diseases caused by bacterial infections by reversing bacteria-induced alterations on cell gene expression profile and coordinating anti-inflammatory and pro-inflammatory responses of an immune system to achieve inhibition of bacterial invasion and elimination of intracellular bacteria.

In one implementation of the present disclosure, MEM prevents and/or treats bacterial meningitis by reducing the number of circulating brain microvascular endothelial cells (cBMECs) in blood.

In one implementation of the present disclosure, MEM prevents and/or treats the diseases caused by multidrug-resistant bacterial infections by performing an antibacterial and/or bactericidal action through neutrophils and neutrophil extracellular traps (NETs).

In one implementation of the present disclosure, MEM is an alpha7 nicotinic acetylcholine receptor (α7nAChR) inhibitor.

In one implementation of the present disclosure, MEM is capable of up-regulating anti-inflammatory genes, including IL-33, IL-18rap, MMP10, and Irs1, and concurrently down-regulating pro-inflammatory genes, including A20, CISH, Ptgds, and ZFP36.

In one implementation of the present disclosure, the bacterial infections are caused by multidrug-resistant bacteria or non-resistant bacteria.

Among them, the multidrug-resistant bacteria include at least one of methicillin-resistant Staphylococcus aureus (MRSA), multidrug-resistant Streptococcus pneumoniae (MDRSP), vancomycin-resistant Staphylococcus aureus (VRSA), multidrug-resistant Mycobacterium tuberculosis (MDR-TB), multidrug-resistant Acinetobacter baumannii (MRAB), and multidrug-resistant E. coli and Klebsiella pneumoniae that carry or do not carry NDM-1 gene.

The non-resistant bacteria include at least one of Staphylococcus aureus, non-resistant Streptococcus pneumoniae, non-resistant Mycobacterium tuberculosis, non-resistant Acinetobacter baumannii, and non-resistant Klebsiella pneumoniae.

In one implementation of the present disclosure, the multidrug-resistant bacterium is methicillin-resistant Staphylococcus aureus (MRSA), and the non-resistant bacterium is Staphylococcus aureus.

In one implementation of the present disclosure, the antibiotic is a beta-lactam antibiotic (e.g., beta-lactam antibiotic penicillin-type or the like). Preferably, the antibiotic is ampicillin.

In a third aspect of the present disclosure, a drug combination used for prevention and/or treatment of diseases caused by bacterial infections is provided, where the combination includes MEM with the chemical structural formula (I) and an antibiotic.

In one of the implementations, the antibiotic is a beta-lactam antibiotic (e.g., beta-lactam antibiotic penicillin-type or the like). Preferably, the antibiotic is ampicillin.

Compared with conventional technologies, the beneficial effects of the present disclosure are as follows:

By applying cell invasion, cell migration, bacteria-infected mice, Next Generation Sequencing, and other techniques, the antibacterial mechanism of MEM is studied using cell models (in vitro) and mouse models (in vivo), the study has found that:

(1) MEM may effectively reverse bacteria-induced alterations on cell gene expression profile. MEM may significantly up-regulate anti-inflammatory genes such as IL-33, IL-18rap, MMP10, and Irs1, and concurrently down-regulate some pro-inflammatory genes such as A20, CISH, Ptgds, and ZFP36. These findings reveal that MEM may achieve inhibition of bacterial invasion and elimination of intracellular bacteria through coordinating the balance in expression of anti-inflammatory genes and pro-inflammatory genes.

(2) MEM may reduce the number of circulating brain microvascular endothelial cells (cBMECs) (biomarkers for neonatal sepsis and meningitis) in blood, blocking the occurrence of bacterial meningitis.

(3) Evidence from experiments using inhibitors and agonists shows that the antibacterial effect of MEM is mediated through inhibition of α7nAChR.

Compared to other drugs, advantages of MEM include (1) MEM may effectively kill intracellular bacteria; (2) MEM has low toxicity and has been on the market for the treatment of moderate-to-severe Alzheimer's disease (AD) for 30 years, proving its safety; and (3) the effects of MEM are mainly mediated through inhibition of α7nAChR to effectively block the three pathogenic hallmark features of bacterial meningitis, providing MEM is highly targeting and efficient.

### DESCRIPTION OF THE DRAWINGS

FIGS. 1A, 1B, 1C, and 1D illustrate memantine (MEM) for enhancing E44-induced neutrophil extracellular trap (NET) formation. Since DNA is a major component of NETs, NET formation is first examined by determining the amount of DNA released from neutrophils. Neutrophils in whole blood are isolated using neutrophil isolation solution Ficoll-Paque (GE Healthcare, USA), 1×10⁵ polymorphonuclear neutrophils (PMNs) are collected and inoculated in a 96-well plate and treated with 100 nM PMA (positive control, Sigma), different concentrations of E44, or E44 (MOI=10) with different concentrations of MEM for 4 hours, PicoGreen (Life Technologies, Invitrogen) working solution is then added to treat each sample for 10 minutes while protecting from light, and a fluorescence value of each sample is measured using a spectrofluorometer. Methods are described in Reference 1. As shown in FIGS. 1A and 1B, although the amount of DNA in the supernatant of neutrophil cultures is increased depending on the concentration of E44, the release of DNA under the effect of E44 is significantly enhanced by the addition of MEM, which is concentration-dependent.

Next, NETosis Assay Kit (Cayman Chemical, USA) is used to detect the production of neutrophil elastase (NE) under treatments of E44 and/or MEM. The methods of neutrophil isolation and preparation are similar to those described previously, and the amount of NE is detected by following kit instructions. The results are similar to the level of DNA release illustrated in FIGS. 1A and 1B, as shown in FIGS. 1C and 1D, the release of NE is promoted by E44. With the addition of MEM, the production of NE under the effect of E44 is significantly increased, and becomes highly concentration-dependent.

FIGS. 2A, 2B, 2C, 2D, and 2E show enhancing bacterial killing effects of neutrophils by implementing the MEM on neutrophils. The bactericidal functions of neutrophils include phagocytosis and NET-mediated killing. Phagocytosis inhibitor, Cytochalasin D (Abcam, USA), is used to distinguish between phagocytosis and NET-mediated killing of neutrophils, and whether MEM may enhance the killing of E44 by neutrophils is determined. Experiments are performed according to the procedures described in Reference 2. Neutrophils (1×10⁶) are inoculated in 24-well plates containing with or without Cytochalasin D, and then added with E44 (MOI=10) and different concentrations of MEM (0, 10, 20, and 50 μM) to treat for 2 hours. The bacteria are collected and spread over Brain Heart Infusion (BHI) agar plates containing rifampin and allowed to culture for 12-16 hours, then the number of bacterial colonies of each sample is counted. FIG. 2A illustrates that MEM may concentration-dependently enhance the killing of E44 by neutrophils, including phagocytosis and NET-mediated killing. Next, whether MEM may enhance NETs on trapping of E44 is examined by using DNase to dissolve DNA. FIG. 2B demonstrates that MEM may concentration-dependently enhance NETs on trapping of E44. Different concentrations of E44 (MOI=1, 5, 50, and 100) are treated with 50 μM of MEM while incubated with PMNs to act as experimental groups, samples without adding MEM are control groups. After 1-hour incubation, the samples are centrifuged, collected, and mounted on microscope slides, which are sealed after DAPI staining, then phagocytosis of PMNs is observed and phagocytized particles are counted under a fluorescence microscope. FIG. 2C indicates that more E44 are phagocytized in E44+MEM groups while comparing to E44 groups. However, early phagocytosis is not accompanied by a significant change in PMN structure when MOI<50. When MOI>50, each PMN on average includes at least 50 E44, a significant nuclear morphological change occurs.

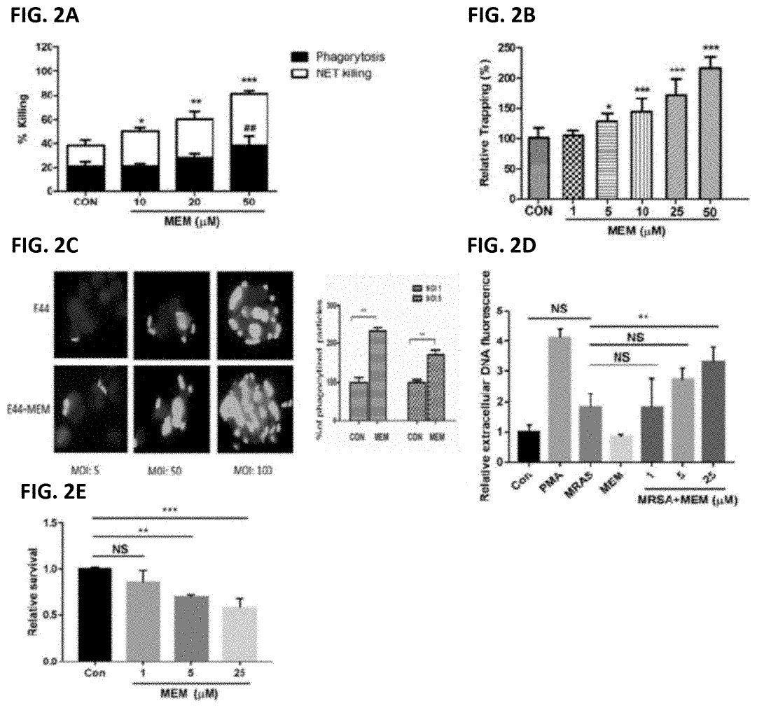

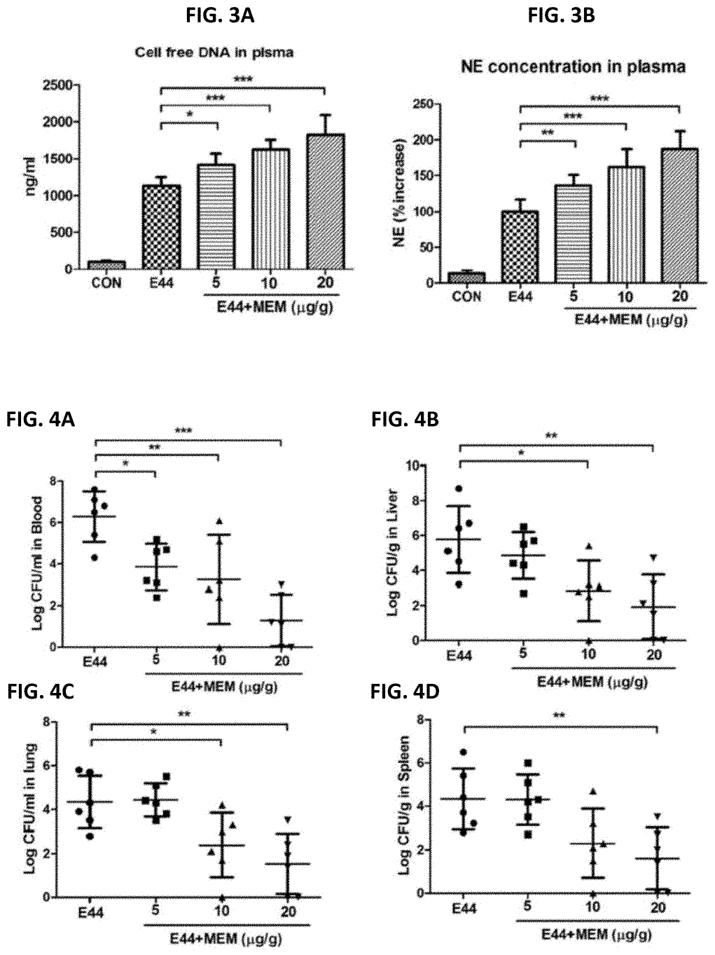

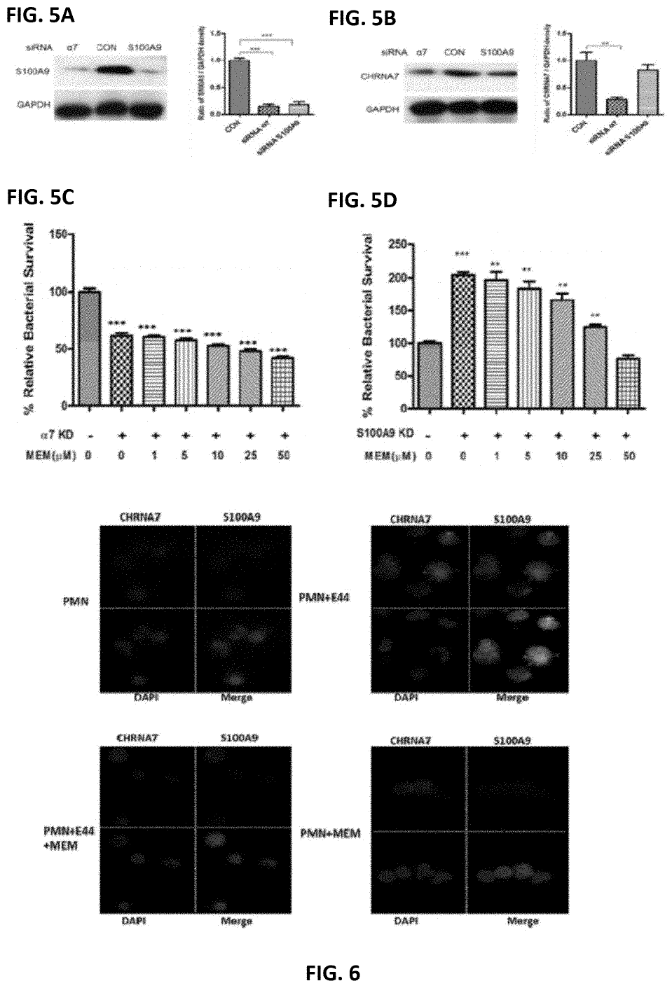

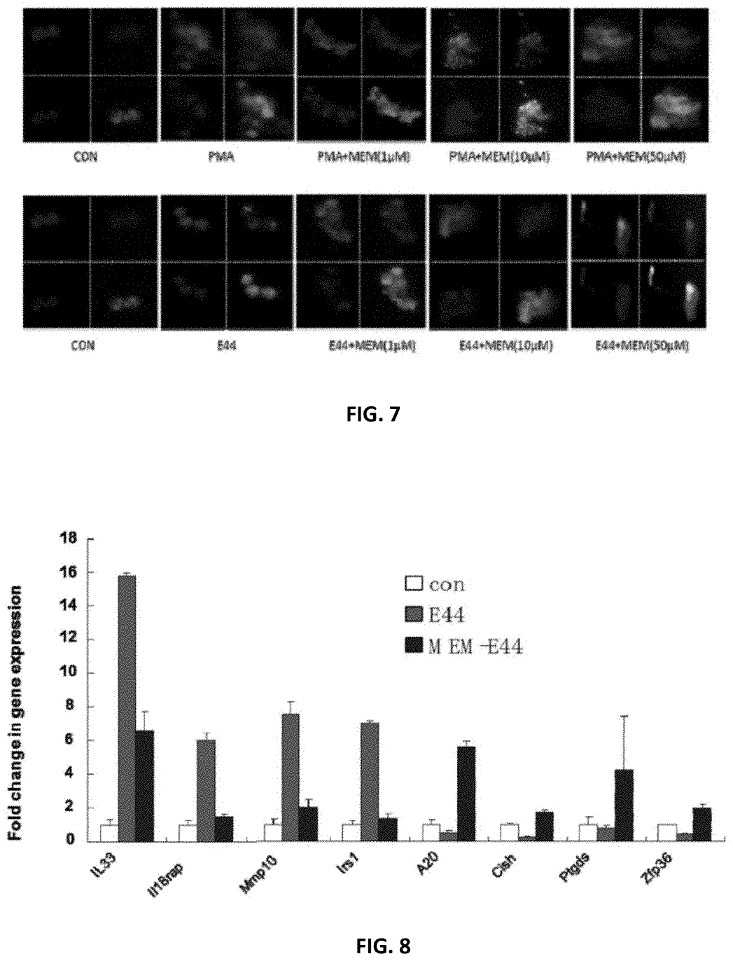

The following discussion includes whether MEM may enhance MRSA-induced NET formation and whether MEM may also enhance killing of MRSA by neutrophils. Experimental procedures of MEM enhancing NET formation and facilitating killing of MRSA by neutrophils are the same as described previously. FIG. 2D shows that MEM may significantly enhance the release of neutrophil DNA, which is similar to E44, MEM may also enhance NET formation under the effects of MRSA. The result of MEM-facilitated bacterial killing experiment reveals that the survival rate of MRSA gradually decreases with increased concentration of MEM (1-25 μM), which is concentration-dependent (FIG. 2E). These findings suggest that MEM may enhance neutrophils to kill MRSA.

FIGS. 3A and 3B show enhancing effects of MEM on NET formation in bacteria-infected mice. 6-8 week-old female C57 mice are injected intraperitoneally with 2×10⁷ E44 or 2×10⁷ E44 plus different concentrations of MEM (5-10 μg/g body weight). After 24 hours, serum is obtained from blood samples collected from hearts, and the amounts of free DNA and neutrophil elastase (NE) in the serum of each mouse are determined using PicoGreen and Mouse PMN Elastase ELISA Kit (R&D Systems) respectively. FIGS. 3A and 3B illustrate that MEM may significantly enhance the productions of free DNA and NE in the blood of E44-infected mice, indicating MEM may enhance NET formation after E44 infection in vivo.

FIGS. 4A, 4B, 4C, and 4D show blocking effects of MEM on the spread of bacteria in bacteria-infected mice. After it is clarified that MEM may enhance NET formation in E44-infected mice. whether MEM may block the spread of E44 in mice is discussed. 6-8 week-old female C57 mice are injected intraperitoneally with 2×10⁷ E44 or 2×10⁷ E44 plus different concentrations of MEM (5-10 μg/g body weight). After 24 hours of E44 infection, the mice are dissected to obtain blood, livers, lungs, and spleens under aseptic condition. The obtained blood, after performing an appropriate serial dilution, is spread over BHI (containing rifampin) agar plates. The obtained livers, lungs, and spleens are precisely weighed, added with pre-cooled sterile PBS in a ratio of 0.1 g/ml, and fully homogenized. The homogenates, after performing an appropriate serial dilution, are spread over BHI (containing rifampin) agar plates. FIGS. 4A-4D illustrate that MEM may significantly block the spread of E44 in mice.

FIGS. 5A, 5B, 5C, and 5D illustrate the influence on phagocytosis by PMNs after gene silencing of CHRNA7 and S100A9. 2×10⁶/ml HL60 cells are treated with 1.3% DMSO for cell stimulation and induction, and cell morphology is observed under an inverted microscope after 4-5 days. Compared to the control group, the cell volume gradually enlarges, granulocyte nuclear shapes such as kidney-shaped (banded) nucleus, segmented (multi-lobed) nucleus, bean-shaped nucleus appear after 48 hours and gradually increase over time. Cell status of each experimental group is identified using Typan Blue Exclusion Test and Wright-Giemsa Stain Method to meet the requirements for cell quality in follow-up studies. Differentiated neutrophils are added with CHRNA7 and S100A9siRNA, respectively, and treated with lipofectamine 2000 in 24-well plates, a supplemental complete cell culture medium is added after 6 hours to incubate for 48 hours, and the expression of CHRNA7 and S100A9 in differentiated HL60 (dHL60) cells is examined. Control siRNA acts as the control group, and GAPDH represents the housekeeping gene. As shown in FIGS. 5A and 5B, results from Western Blot reveal that the gene expression of CHRNA7 and S100A9 in the dHL60 cells is successfully down-regulated, with down-regulation efficiency up to 80%, which may be used in subsequent experiments. When CHRNA7 is down-regulated, the expression of S100A9 is also significantly decreased (FIG. 5A). However, when S100A9 is down-regulated, the expression of CHRNA7 is not changed much as compared to the control group, indicating the expression of S100A9 is regulated by CHRNA7 (FIG. 5B).

After the gene expression of CHRNA7 and S100A9 in the dHL60 cells is down-regulated using siRNA technique, the experiment on killing of E44 by phagocytosis of PMN is performed. Samples are prepared by the dHL60 cells and E44 in a ratio of 1:5 and allowed to interact for 1 hour. The samples are then diluted to spread over Luria Broth (LB) agar plates and cultured overnight to detect the effect of MEM on the ability of PMN to phagocytize E44 after down-regulating the gene expression of CHRNA7 and S100A9. Results are expressed as relative survival rate of E44, as shown in FIG. 5C, the survival rate of E44 decreases after the gene expression of CHRNA7 is down-regulated when comparing to the cells in control group, whereas the effect of MEM, with dose up to 50 μM, on phagocytizing E44 by PMNs is not obvious. As illustrated in FIG. 5D, the survival rate of E44 increases after the gene expression of S100A9 is down-regulated when comparing to the cells in control group, which has statistical differences.

FIG. 6 illustrates effects of MEM on the interaction between CHRNA7 and S100A9 in phagocytic mechanism of PMN. The next step is to further examine whether alpha7 nicotinic acetylcholine receptor (α7nAChR) is associated with antimicrobial protein S100A9 during the process of phagocytizing E44 by PMNs. PMNs isolated from peripheral whole blood are treated with MEM while incubated with E44, untreated PMNs represent the control group. Next, an immunofluorescence staining test is performed on the samples as follows: a primary antibody is added to incubate with α7nAChR and S100A9 overnight at 4° C., and then the corresponding FITC and PE-conjugated secondary antibodies are added to incubate for 1 hour at room temperature, after mounting the samples on microscope slides with DAPI sealing, positioning and expression of α7nAChR and S100A9 are observed under a fluorescence microscope. Compared to the control group, S100A9 (red) is redistributed to the cell edge and area around the nucleus (FIG. 6) after infected with E44, it is observed that α7nAChR (green) and S100A9 (red) are located in the same position with presenting of colocalization, and the expression of both is elevated, α7nAChR (green) and S100A9 (red) also appear to have a high degree of colocalization(R close to 1) after ImageJ software analysis. Compared to the E44 groups, the expression of α7nAChR and S100A9 is significantly decreased after the treatment of MEM, and the degree of colocalization on both is remarkably reduced. These findings explain that α7nAChR and S100A9 are dissociated and reduced in colocalization after the stimulation of MEM.

FIG. 7 shows the impacts of MEM on inducing an increase in expression of antimicrobial enzyme MPO and antimicrobial protein S100A9. After the purity of PMNs isolated from whole blood reaches 95%, the PMNs are treated with PMA and pathogenic E44 respectively and then stimulated by different doses of MEM, immunofluorescence staining is carried out subsequently. In order to prove NET formation, each set of samples is stained directly with antibodies MPO (green) and S100A9 (red), after an addition of DAPI (blue), changes in cell morphology are observed under a fluorescence microscope. As shown in FIG. 7, most nuclei in the control group remain in a segmented nuclear structure. In the PMA groups and E44 groups treated with MEM, a large number of MPO (green), S100A9 (red), and DNA (blue) are formed, nuclear structures of cells are destroyed, and chromatin is decondensed to viscous webs and released into the extracellular space, indicating NET formation.

FIG. 8 illustrates the effects of MEM on reversing E44-induced alterations on gene expression, making it similar to the control group. MEM may significantly up-regulate anti-inflammatory genes such as IL-33, IL-18rap, MMP10, and Irs1, and concurrently down-regulate some pro-inflammatory genes such as A20, CISH, Ptgds, and ZFP36.

### DETAILED DESCRIPTION

In order to facilitate understanding of the present disclosure, the following will refer to the relevant drawings for a more comprehensive description of the present disclosure. The implementations of the present disclosure are shown in the drawings. However, the present disclosure may be implemented in many different forms and is not limited to the implementations described herein. On the contrary, the purpose of providing these implementations is to make a more thorough and comprehensive understanding on the content of the present disclosure.

Unless otherwise defined, all the technical and scientific terms used herein are the same as those commonly understood by technical personnel who belong to the technical field of the present invention. The terms used in the description of the present disclosure in this application are intended only for the purpose of describing a specific implementation and are not intended to limit the present disclosure. The term “and/or” used herein includes any and all combinations of one or more related listed items.

In the present disclosure, cell models (in vitro) and mouse models (in vivo) are used to study the effect of memantine (MEM), either by MEM alone or in combination with another agent, on E44-invaded brain microvascular endothelial cells (BMECs), examine the effect of MEM on E44-induced gene expression profile alterations in BMECs by Next Generation Sequencing, test the changes in the number of circulating brain microvascular endothelial cells (cBMECs) (biomarkers for neonatal sepsis and meningitis) in blood, and reveal the antibacterial effect of MEM based on the effects of MEM on meningitis and bacteremia in E44-infected mice, which provide the experimental basis for antibacterial applications with MEM. The protocol is prepared as follows:

1. Experimental Materials

1.1 Cell Strain Human Brain Microvascular Endothelial Cells (HBMECs) and Mouse Microvascular Endothelial Cells (BMECs)

1.2 Chemicals and Reagent: RPMI-1640 medium (Gibco, USA), 10% fetal bovine serum (Gibco, USA), 1% non-essential amino acids solution (Gibco, USA), nicotine and MEM (Sigma, USA), Dynabeads M-450, Fura-2AM, Pluronic-127 (Invitrogen, USA), Ulex europaeus I (UEA I) (Vector, USA), PBS, gentamicin, and ampicillin.

1.3 Instruments: an optical microscope (fluorescence microscope), 24-well cell culture plates, Transwell polycarbonate membrane inserts (Corning Costar), and a spectrophotometer.

2. Methods

2.1 Isolation, Culture, and Identification of Mouse Microvascular Endothelial Cells (BMECs)

According to manufacturer's instructions, Dynabeads M-450 solution with a final concentration of 4×10⁸ beads/ml is prepared by Hank's Balanced Salt Solution. BMECs in brain tissues and blood are isolated using UEA I. The BMECs from the brain tissues are cultured with RPMI-1640 medium containing 10% fetal bovine serum and identified using CD146 and Mfsd2a antibodies.

2.2 Blocking Effects of MEM on Bacterial Invasion Experiment

HBMECs are inoculated in 24-well plates, waiting for the cells to be confluent, and cultured using the corresponding concentrations of MEM, nicotine, and kynurenic acid for 1 or 24 hours. After treatments of the drugs, the HBMECs are washed with PBS 3 times and then infected with 10⁷ E44. After HBMEC monolayers are cultured for 1.5 hours at 37° C., excess bacteria are washed away using PBS. Next, the HBMEC monolayers are cultured in a medium containing gentamicin to kill extracellular bacteria and then lysed to release intracellular bacteria, and the number of invasive bacteria in the cells is counted.

2.3 Neutrophil Transmigration Experiment

HBMECs are inoculated in upper compartments of 24-well plates with Transwell inserts, waiting for the cells to be confluent, and added with the corresponding drugs such as MEM and nicotine to treat for 2 hours. 10⁵ bacteria are inoculated in lower compartments, after 2 hours, 10⁶ neutrophils are added to the upper compartments, and the numbers of neutrophils in the lower compartments are counted after 4 hours.

2.4 Inhibition of MEM on Bacteremia and Meningitis in Mice

10-day-old mice are given with different concentrations of MEM by intraperitoneal injection, 12 hours later, the mice are inoculated with 2×10⁵ E44 intraperitoneally. After 15 hours, the mice are anesthetized to collect blood and cerebrospinal fluid (CSF), and bacterial counts in the blood and CSF are determined.

2.5 Effects of MEM on Gene Expression Profile During the Process of E44 Invasion in BMECs

Mouse BMECs are inoculated in 6-well plates, waiting for the cells to be confluent, and divided into four groups including a control group, an E44 group, a MEM-treated group, and an E44+MEM group. After the mouse BMECs are infected with E44 for 0.5 hours, the cells are collected to extract RNA, which is sent to Hua-Da Company (China) for Next Generation Sequencing analysis.

3. Results

3.1 MEM Enhancing Bacteria-Induced NET Formation

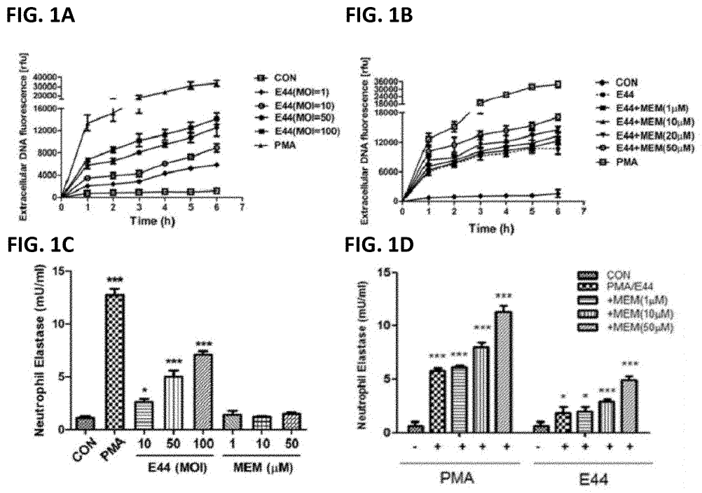

As shown in FIGS. 1A and 1B, MEM may, depending on the concentration, enhance E44-induced NET formation, the effects of MEM has shown the same concentration dependence whether in different numbers of bacteria (FIG. 1A) or different doses of drug (FIG. 1B). Although the amount of DNA in the supernatant of neutrophil cultures is increased depending on the concentration of E44, the release of DNA under the effect of E44 is significantly enhanced by the addition of MEM, which is highly concentration-dependent. The results demonstrated in FIGS. 1C and 1D are similar to the level of DNA release described above (FIGS. 1A and 1B). The release of neutrophil elastase (NE) is promoted by E44. With the addition of MEM, the production of NE under the effect of E44 is significantly increased, and becomes highly concentration-dependent.

3.2 MEM Enhancing NET-Mediated Trapping and Killing of Bacteria

As shown in FIG. 2A, MEM may concentration-dependently enhance the killing of E44 by neutrophils, including phagocytosis and NET-mediated killing. As illustrated in FIG. 2B, MEM may concentration-dependently enhance NETs on trapping of E44. Different concentrations of bacteria are treated with the same amount of MEM while incubated with PMNs to act as experimental groups, samples without adding MEM are control groups. As demonstrated in FIG. 2C, more E44 are phagocytized in E44+MEM groups while comparing to E44 groups. However, early phagocytosis is not accompanied by a significant change in PMN structure when MOI<50. When MOI>50, each PMN on average includes at least 50 E44, a significant nuclear morphological change occurs. The following discusses whether MEM may enhance MRSA-induced NET formation and whether MEM may also enhance killing of MRSA by neutrophils. Experimental procedures of MEM enhancing NET formation and facilitating killing of MRSA by neutrophils are the same as described previously. As shown in FIG. 2D, MEM may significantly enhance the release of neutrophil DNA, which is similar to E44, MEM may also enhance NET formation under the effect of MRSA. The result of MEM-facilitated bacterial killing experiment reveals that the survival rate of MRSA gradually decreases with increased concentration of MEM (1-25 μM), which is concentration-dependent (FIG. 2E). These findings suggest that MEM may enhance neutrophils to kill MRSA.

3.3 MEM Enhancing NET Formation in Bacteria-Infected Mice

After mice are infected with bacteria, serum is obtained from blood samples collected from hearts, and the amounts of free DNA and neutrophil elastase (NE) in the serum of each mouse are determined, respectively. As shown in FIGS. 3A and 3B, MEM may significantly enhance the productions of free DNA and NE in the blood of bacteria (E44)-infected mice, indicating MEM may enhance NET formation after bacterial infection in vivo.

3.4 MEM may Blocking Spread of E44 in Mice

After confirming that MEM may enhance NET formation in bacteria-infected mice, whether MEM may block the spread of bacteria in mice is discussed. Female C57 mice (6-8 week-old) are injected intraperitoneally with E44 (2×10⁷ CFU) or the same amount of bacteria plus different concentrations of MEM (5-10 μg/g body weight). After 24 hours of bacterial infection, the mice are dissected to obtain blood, livers, lungs, and spleens under aseptic condition for bacterial count determination. As shown in FIGS. 4A-4D, MEM may significantly block the spread of bacteria (E44) in mice.

3.5 Influence on Phagocytosis by PMNs After Gene Silencing of CHRNA7 and S100A9

In order to clarify the influence on phagocytosis by PMNs after acne expression knockdown (gene silencing) of CHRNA7 and S100A9, after HL60 cells are treated with 1.3% DMSO to stimulate and induce cell differentiation, CHRNA7 and S100A9siRNA are added to the differentiated HL60 (dHL60) cells respectively to perform gene expression knockdown, and the expression of CHRNA7 and S100A9 in the dHL60 cells is examined. Control siRNA acts as the control group, and GAPDH represents the housekeeping gene. As shown in FIGS. 5A and 5B, results from Western Blot reveal that the gene expression of CHRNA7 and S100A9 in the dHL60 cells is successfully down-regulated, with down-regulation efficiency up to 80%, which may be used in subsequent experiments. When CHRNA7 is down-regulated, the expression of S100A9 is also significantly decreased (FIG. 5A). However, when S100A9 is down-regulated, the expression of CHRNA7 is not changed much as compared to the control group, indicating the expression of S100A9 is regulated by CHRNA7 (FIG. 5B). After the gene expression of CHRNA7 and S100A9 in the dHL60 cells is down-regulated using siRNA technique, the experiment on killing of bacteria by phagocytosis of PMN is performed, and results are expressed as relative survival rate of bacteria. As shown in FIG. 5C, the survival rate of bacteria decreases after the gene expression of CHRNA7 is down-regulated when comparing to the cells in control group, whereas the effect of MEM, with dose up to 50 μM, on phagocytizing bacteria by PMNs is not obvious. As illustrated in FIG. 5D, the survival rate of bacteria increases after the gene expression of S100A9 is down-regulated when comparing to the cells in control group, which has statistical differences.

3.6 Interaction Between CHRNA7 and S100A9 in Phagocytic Mechanism of PMN

The study further analyzed whether alpha7 nicotinic acetylcholine receptor (α7nAChR) is associated with antimicrobial protein S100A9 during the process of phagocytizing bacteria by PMNs. PMNs isolated from peripheral whole blood are treated with MEM while incubated with bacteria, untreated PMNs represent the control group. Next, an immunofluorescence staining test is performed on the samples to observe the positioning and expression of α7nAChR and S100A9 under a fluorescence microscope. Compared to the control group, S100A9 (red) is redistributed to the cell edge and area around the nucleus (FIG. 6) after infected with bacteria, α7nAChR (green) and S100A9 (red) are located in the same position with presenting of colocalization, and the expression of both is elevated, α7nAChR (green) and S100A9 (red) also appear to have a high degree of colocalization (R close to 1) after ImageJ software analysis. Compared to the bacteria groups, the expression of α7nAChR and S100A9 is significantly decreased after the treatment of MEM, and the degree of colocalization on both is remarkably reduced. These findings explain that α7nAChR and S100A9 are dissociated and reduced in colocalization after the stimulation of MEM.

3.7 MEM may Induce an Increase in Expression of Antimicrobial Enzyme MPO and Antimicrobial Protein S100A9

After the purity of PMNs isolated from whole blood reaches 95%, the PMNs are treated with PMA and pathogenic E44 respectively and then stimulated by different doses of MEM, immunofluorescence staining is carried out subsequently. In order to prove NET formation, each set of samples is stained directly with antibodies MPO (green) and S100A9 (red), after an addition of DAPI (blue), changes in cell morphology are observed under a fluorescence microscope. As shown in FIG. 7, most nuclei in the control group remain in a segmented nuclear structure. In the PMA groups and E44 groups treated with MEM, a large number of MPO (green), S100A9 (red), and DNA (blue) are formed, nuclear structures of cells are destroyed, and chromatin is decondensed to viscous webs and released into the extracellular space, indicating NET formation.

3.8 Effect of MEM on Gene Expression Profile During the Process of E44 Invasion in HBMECs

In order to intensely analyze the mechanism of MEM on inhibition of bacterial invasion and elimination of intracellular bacteria, mouse BMECs are divided into four groups including a control group, an E44 group, a MEM-treated group, and an E44+MEM group. After the mouse BMECs are infected with E44 for 0.5 hours, the cells are collected to extract RNA, which is sent to Hua-Da Company (China) to analyze alterations on gene expression profile using Next Generation Sequencing and look for differentially expressed genes, a total of 978 differentially expressed genes are found. As can be seen from FIG. 8, MEM may reverse E44-induced alterations on gene expression, making it similar to the control group. MEM may significantly up-regulate anti-inflammatory genes such as IL-33, IL-18rap, MMP10, and Irs1, and concurrently down-regulate some pro-inflammatory genes such as A20, CISH, Ptgds, and ZEP36. FIG. 8 shows the specific folds of each gene expression relative to the control group. These findings demonstrate that MEM achieves inhibition of bacterial invasion and elimination of intracellular bacteria through coordinating the balance in expression of anti-inflammatory genes and pro-inflammatory genes.

3.9 MEM and Ampicillin Synergistically Killing E44 in HBMECs

In order to further clarify the inhibitory range of MEM on E44 proliferation in HBMECs, whether a combination of MEM and an antibiotic is superior to either medication alone is determined. HBMECs are pre-treated with 25 μM of MEM for 12 hours and then infected with E44 for another 1.5 hours, the E44-infected HBMECs are treated with 5-50 μg/ml ampicillin alone and ampicillin in combination with MEM respectively, the results suggest that the combination of ampicillin and MEM provides a synergistic effect to inhibit the survival of E44 in HBMECs. The effect of the combined therapy is greater than either medication given alone. Similar results are obtained in experiments using mouse models.

Based on the NET experiment, MEM also has an antibacterial and/or bactericidal effect on other multidrug-resistant bacteria (such as multidrug-resistant Streptococcus pneumoniae, vancomycin-resistant Staphylococcus aureus, multidrug-resistant Mycobacterium tuberculosis, multidrug-resistant Acinetobacter baumannii, and multidrug-resistant E. coli and Klebsiella pneumoniae that carry or do not carry NDM-1 gene), and the antibacterial and/or bactericidal mechanism is the same as the one that MEM eliminates methicillin-resistant Staphylococcus aureus.

The implementations described above represent only a few implementations of the present disclosure, descriptions are more specific and detailed, but cannot be understood as a limitation on the scope of the present disclosure. It should be pointed out that a person of ordinary skill in the art, without departing from the concept of the present invention, may also make a number of deformations and improvements, these are the scope of protection of the present disclosure.

### CITATION LIST

Cited Reference 1: Capsule and D-alanylated lipoteichoic acids protect Streptococcus pneumoniae against neutrophil extracellular traps. Cell Microbiol., 2007 May; 9 (5):1162-71. PMED:17217430.

Cited Reference 2: Cai S, Qiao X, Feng L, Shi N, Wang H, Yang H, Guo Z, Wang M, Chen Y, Wang Y, Yu H. Python Cathelicidin CATHPb1 Protects against Multidrug-Resistant Staphylococcal Infections by Antimicrobial-Immunomodulatory Duality. J Med Chem. 2018 Mar. 8; 61(5):2075-2086. PMID:29466000.

Cited Reference 3: Schechter M C, Buac K, Adekambi T, Cagle S, Celli J, Ray S M, Mehta C C, Rada B, Rengarajan J. Neutrophil extracellular trap (NET) levels in human plasma are associated with active TB. PLoS One. 2017 Aug. 4; 12(8):e0182587. PMID:28777804.

What is claimed is:

1. A method comprising:
applying memantine (MEM) in preparation of one or more drugs for preventing or treating a disease caused by a bacterial infection,
wherein a chemical structural formula of the MEM is shown as (I):

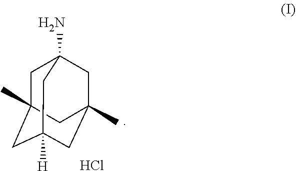

2. The method of claim 1, wherein the disease is caused by an intracellular multidrug-resistant bacterial infection.

3. The method of claim 2, wherein a bacterium causing the intracellular multidrug-resistant bacterial infection is E. coli E44.

4. The method of claim 1, wherein the disease is bacterial meningitis or neonatal sepsis.

5. The method of claim 1, wherein the MEM prevents or treats the disease by reversing bacteria-induced alterations on a cell gene expression profile and coordinating anti-inflammatory and pro-inflammatory responses of an immune system to achieve inhibition of bacterial invasion and elimination of intracellular bacteria.

6. The method of claim 4, wherein the MEM prevents or treats the bacterial meningitis by reducing the number of circulating brain microvascular endothelial cells (cBMECs) in blood.

7. The method of claim 1, wherein the MEM prevents or treats the disease by performing an antibacterial or bactericidal action through neutrophils and neutrophil extracellular traps (NETs).

8. The method of claim 1, wherein the MEM is an alpha7 nicotinic acetylcholine receptor (α7nAChR) inhibitor.

9. The method of claim 5, wherein the MEM is capable of up-regulating anti-inflammatory genes including IL-33, IL-18rap, MMP10, and Irs1, and concurrently down-regulating pro-inflammatory genes including A20, CISH, Ptgds, and ZFP36.

10. The method of claim 1, wherein:
the bacterial infection is caused by one or more multidrug-resistant bacteria or non-resistant bacteria,
the multidrug-resistant bacteria include at least one of:
methicillin-resistant Staphylococcus aureus (MRSA),
multidrug-resistant Streptococcus pneumoniae (MDRSP),
vancomycin-resistant Staphylococcus aureus (VRSA),
vancomycin-resistant Enterococcus (VRE),
multidrug-resistant Mycobacterium tuberculosis (MDR-TB),
multidrug-resistant Acinetobacter baumannii (MRAB),
multidrug-resistant E. coli that carry or do not carry NDM-1 gene, and
multidrug-resistant Klebsiella pneumoniae that carry or do not carry NDM-1 gene,
the non-resistant bacteria include at least one of:
non-resistant Staphylococcus aureus,
non-resistant Streptococcus pneumoniae,
non-resistant Mycobacterium tuberculosis,
non-resistant Acinetobacter baumannii, and
non-resistant Klebsiella pneumoniae.

11. The method of claim 1, further comprising:
combining the MEM with an antibiotic in the preparation of the one or more drugs.

12. The method of claim 11, wherein the antibiotic is a beta-lactam antibiotic.

13. The method of claim 12, wherein the beta-lactam antibiotic is ampicillin.

14. A combination of drugs used for prevention or treatment of a disease caused by a bacterial infection, the combination comprising:
memantine (MEM) having a chemical structural formula shown as (I): 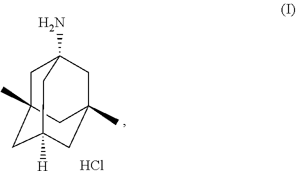
and
an antibiotic.

15. The combination of drugs of claim 14, wherein the antibiotic is a beta-lactam antibiotic.

16. The combination of drugs of claim 15, wherein the beta-lactam antibiotic is ampicillin.
